(19) United States
(12) Patent Application Publication
(10) Pub. No.: US 2022/0040115 A1
(43) Pub. Date: Feb. 10, 2022

(54) PHARMACEUTICAL COMPOSITIONS AND DELIVERY SYSTEMS FOR PREVENTION AND TREATMENT OF CANDIDIASIS

(71) Applicant: INDIAN INSTITUTE OF TECHNOLOGY HYDERABAD, Sangareddy (IN)

(72) Inventors: Mudrika Khandelwal, Sangareddy (IN); Shivakalyani Adepu, Sangareddy (IN)

(21) Appl. No.: 17/276,478

(22) PCT Filed: Sep. 17, 2019
(86) PCT No.: PCT/IB2019/057802

§ 371 Date: Mar. 15, 2021

(30) Foreign Application Priority Data
Sep. 17, 2018 (IN) 201841034939

(51) Int. Cl. A61K 9/50; A61K 47/34; A61K 47/24; A61K 31/015; A61K 31/085; A61K 31/05

(52) U.S. Cl. CPC A61K 9/5031; A61K 47/34; A61K 47/24; A61K 9/5068; A61K 31/085; A61K 31/05; A61K 31/015

(57) ABSTRACT

Pharmaceutical composition and delivery system for prevention and treatment of candidiasis featuring a composition of thymol oil, eugenol oil, and/or carvacrol oil encapsulated in microcapsules, with the microcapsules embedded in a medicated microbial cellulosic matrix. The cellulosic matrix may in turn be embedded in or form a conveyance material, such as a panty liner, a mucoadhesive patch, or chewing gum.

### PRIORITY CLAIM

This application claims the benefit of priority to PCT Application number PCT/IB2019/057802, filed on Sep. 17, 2019, which in turn claims the benefit of priority of Indian Provisional Patent Application number 201841034939, filed on Sep. 17, 2018, both of which are hereby incorporated by reference herein in their entirety.

### FIELD OF INVENTION

The present invention pertains to pharmaceuticals and delivery systems for the prevention and treatment of candidiasis.

### BACKGROUND

Candidiasis, also called thrush is a condition caused by an overgrowth of Candida species on the lining of the mouth, skin or vagina. Depending on the site of infection, the pathological conditions are termed as oral candidiasis, cutaneous candidiasis or vaginal candidiasis. The most common amongst the Candida species causing candidiasis is Candida albicans. The other common species involved are Candida glabrata and Candida tropicalis. Candidiasis can lead to inflammation, intense itchiness, local discomfort, soreness at the site of infection. Oral candidiasis additionally causes an altered taste sensation and dysphagia, resulting in reduced food intake.

Current approaches in the management of candidiasis involve administration of antifungal azoles in various formulations such as creams, ointments, tablets, suppositories, gels, lozenges, mucoadhesive patches or mixtures. For the treatment of mild to moderate infections, an antifungal medicine applied on the site of the infection. Anti-fungal medications include clotrimazole, miconazole, nystatin, butoconazole, terconazole, and tioconazole. For severe infections, the treatment is usually administration of fluconazole which may be combined with other types of antifungal medicines administered intravenously.

The above medications suffer from the following drawbacks:
1. The administration of antifungal medicines is associated with major side-effects such as nausea, vomiting, diarrhea, headache and dizziness.
2. For vaginal candidiasis, the most common side effect experienced are burning, itching, abdominal cramps, irritation and allergic reactions.
3. The medications are often hydrolyzed by the lysozyme, an enzyme present in oral as well as vaginal mucosa.
4. For oral candidiasis, the bioavailability of topical medicines applied on the lining of the mouth is extremely low leading to suboptimal therapeutic effect. The retention time is low due to salivation and consumption of food and water. Finally, the antifungal medicines are often bitter in taste which leads to ineffectiveness in administration, especially in pediatric and geriatrics administration.
5. Any sort of antimicrobial drugs often suffers from antimicrobial resistance.
6. Antifungal medicines may damage oral or vaginal mucosa.

As an alternative therapeutic approach, essential oils and their active ingredients have been reported to have excellent anti-fungal and antibacterial activity. The essential oils and their active ingredients additionally have an emollient effect on the oral mucosa, vaginal mucosa and cutaneous membrane. Further, they enhance the healing process because of their splendid antioxidant properties. However, there are three major drawbacks in using these essential oils and their active ingredients for the management of candidiasis:
1. Usage of a single essential oil is an inferior therapeutic intervention as compared to a synergistic combination of oil.
2. The essential oils are unstable and extremely sensitive to temperature, light and pH.
3. Any formulation or chemical modifications often lead to diminished activity of these oils.

Therefore, there is a need to formulate synergistic compositions of essential oils which can be used for effective prevention and treatment of candidiasis.

For oral as well as vaginal candidiasis, the inventors have identified that microbial cellulose constitutes a valuable delivery system for effective delivery of the essential oils. Microbial cellulose provides huge advantages over conventional drug delivery systems, which includes the fast onset of action, sustained release of drugs and high bioavailability, higher compliance (easy and discreet administration) and ready for use as a point of care solution. Additionally, the cellulosic matrix can be modified as drug delivery system. Cellulosic matrix modified as chewing gum has high acceptance by children. However, there have been no attempts so far for effective delivery of essential oil compositions for prophylactic or therapeutic treatment of candidiasis.

The inventors have identified the above challenges and have addressed the same by preparing compositions comprising essential oils and excipients, essentially aimed at obtaining synergistic effects for prevention and treatment of different types of candidiasis, including, oral, vaginal and cutaneous candidiasis. Further, the inventors have envisaged a unique approach for effective delivery of the active components at the site of the infection by encapsulating the essential oils or their active ingredients in microcapsules and embedding them in the cellulosic matrix. The cellulosic matrix can be modified for chewing. The cellulosic matrix can also be used as a transdermal and mucoadhesive patch. The cellulosic matrix enables sustained and controlled release of the essential oils, which leads to high efficacy in prophylaxis and treatment of candidiasis.

Thus, the present invention overcomes the problems of the prior art to solve a long-standing problem of providing synergistic essential oil compositions and medicated cellulosic matrix for effective management of candidiasis.

### SUMMARY OF THE INVENTION

### Technical Problem

The technical problem to be solved in this invention is effective, inexpensive, safer and non-invasive prevention and management of candidiasis.

### Solution to the Problem

The problem has been solved by a multi-dimensional approach involving:
1. Developing synergistic formulations comprising essential oils or their active ingredients and, optionally excipients;
2. Devising a novel delivery strategy by encapsulating the essential oils in microcapsules; and
3. Loading the microcapsules by embedding them in a microbial cellulosic matrix.

### OVERVIEW OF THE INVENTION

The invention provides for preparation of synergistic essential oil compositions for management of candidiasis, wherein the essential oils are selected from a group comprising thymol oil, eugenol oil, carvacrol oil or their active ingredients.

A further aspect of the invention provides for methods pertaining to encapsulation of essential oils in microcapsules.

Another aspect of the invention provides for medicated microbial cellulosic matrix in which the essential oil microcapsules or their active ingredients are embedded. The cellulosic matrix may optionally comprise excipients. The matrix may be further modified for various uses such as chewing gums, as mucoadhesive and transdermal patches, as liners for female hygiene and the like.

### BRIEF DESCRIPTION OF THE FIGURES

FIG. 1, FIG. 2 and FIG. 3 exhibit the inhibitory effect of the oil compositions of the present invention on Candida albicans.

FIG. 4 represents the positive and negative controls for the studies.

FIG. 5 and FIG. 6 depicts a schematic process for the preparation of microcapsules with oil composition.

FIG. 7 depicts the optical microstructure of microcapsules PLA_{1.5}Pol_{5.0} and PLA_{1.5}Pol_{2.5}.

FIG. 8 depicts the optical microstructure of microcapsules PLA_{3.0}Pol_{5.0} and PLA_{3.0}Pol_{2.5}.

FIG. 9 depicts the results of the DLS microscopic studies on microcapsules.

FIGS. 10 and 11 depict Scanning Electron Microscopic studies to check the embedding of the oil microcapsules in microbial cellulosic matrix.

FIG. 12 depicts the results of the FTIR studies conducted on microcapsule loaded cellulosic matrix.

FIG. 13 and FIG. 14 depicts the results of in vitro drug release studies in simulant vaginal fluid (SVF).

FIG. 15 shows the antifungal activity of the microcapsule loaded cellulosic matrices and microcapsules after 24 hrs of incubation.

FIG. 16 shows the antifungal activity of the microcapsule loaded cellulosic matrices and microcapsules after 48 hrs of incubation.

FIG. 17 shows the antifungal activity of the microcapsule loaded cellulosic matrices and microcapsules after 72 hrs of incubation.

FIG. 18 shows the antifungal activity of the positive and negative controls used in the studies.

FIG. 19, FIG. 20 and FIG. 21 depict time-kill curve analysis of oil compositions, microcapsules and bacterial cellulose loaded microcapsules.

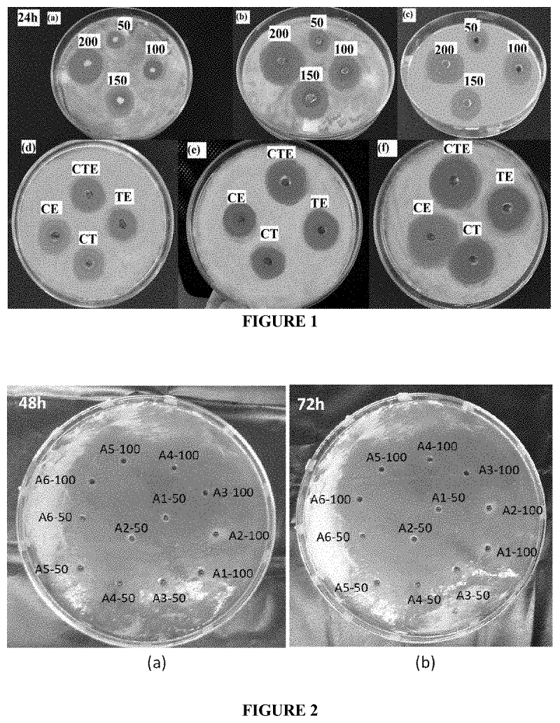

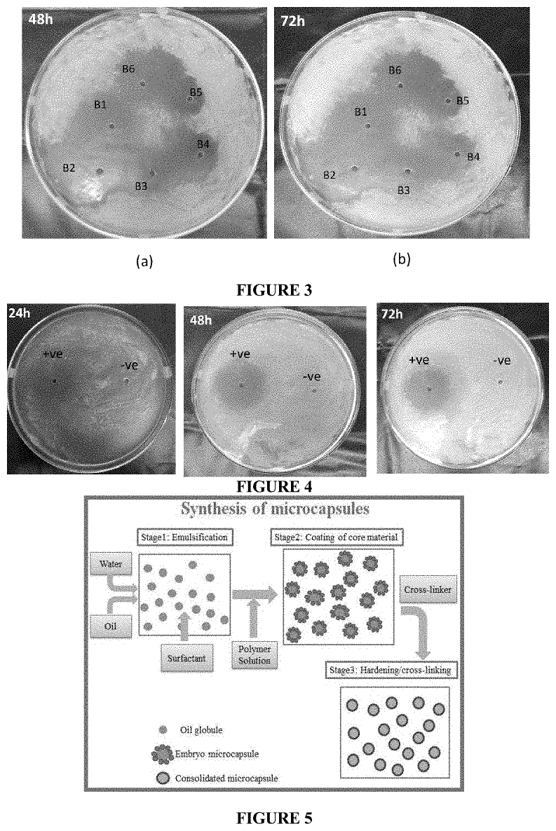

### DETAILED DESCRIPTION OF THE INVENTION

The present invention discloses compositions, methods and delivery systems for effective management of candidiasis. In particular, the invention discloses novel medicated microbial cellulosic matrix for effective prophylaxis and treatment of candidiasis.

For the first time, the inventors have devised a synergistic composition comprising essential oils for effective therapeutic intervention for candidiasis. The inventors have further devised a delivery system by preparing microcapsules of the synergistic oil compositions. Further, the microcapsules have been loaded in a cellulosic matrix for creating a novel drug delivery system.

The present invention represents an advancement over the existing methods for effective management of candidiasis. The advances are characterized by the following features:
(a) Affordable: The compositions and cellulosic matrices developed are highly inexpensive, safe and can be afforded even by people in developing and least-developed nations.
(b) Sensitive and Specific: The compositions and medicated matrices developed are highly sensitive and very specific for management of various bacterial pathogenic conditions, including candidiasis. The embodiments do not have any recorded adverse effects.
(c) User-friendly: The compositions and cellulosic matrices developed represent point of care and over the counter solutions for management of oral and vaginal candidiasis. The instructions for administration are minimal. Further, the matrices modified for oral candidiasis can be administered even to children.
(d) Rapid and Robust: The compositions and medicated matrices show immediate positive results. Further, the medicated matrices do not require special storage conditions.
(e) Delivery to those who need it: The compositions and medicated matrices are extremely cheap and easy to transport even to rural and semi-urban areas, where there is a lack of proper medical infrastructure.

The inventive approach used in the present invention has led to the development of compositions and delivery systems which would help the patients for effective management of candidiasis.

Unless defined otherwise, all technical and scientific terms used herein have the same meaning as commonly understood by one of ordinary skill in the art to which the methods belong. Although any composition and delivery systems similar or equivalent to those described herein can also be used in the practice or testing of the methods and compositions, representative illustrative methods and compositions are now described.

Where a range of values is provided, it is understood that each intervening value between the upper and lower limit of that range and any other stated or intervening value in that stated range, is encompassed within by the methods, compositions and delivery systems. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods, compositions and delivery systems.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As used herein, the term “essential oil” or “oil” encompasses all botanical oils, lipids and active ingredient present in such oils, polyphenols, aldehydes, terpenes and lipids, as described herein. The term may also mean the individual active ingredient of the botanical oils and lipids which can be defined chemically and synthesized using commercially available reagents, without the use of natural products or extracts.

As used herein, the term “drug delivery system” encompasses systems which can be modified and used as a microencapsulated formulation, buccal patch, chewing gum, transdermal patch, mucoadhesive system and the like.

As used herein, the term “microcapsule” refers to particles that have a shell component and a core component wherein the shell component (also known as shell material or capsule shell) encloses the core component, which may be a single core or comprise numerous cores dispersed among the shell material as a matrix. As used herein, the “core material” of the microcapsules of the present material comprises at least two essential oils. As used herein, the “shell material” or “capsule shell” of the microcapsules comprises a principal component selected from the following materials: chitosan, polylactide (PLA), Polymethyl methacrylate (PMMA), poly(N-isopropylmethacrylamide), (PNIPAM) and alginates.

As used herein, the term “microbial cellulosic matrix” or “bacterial cellulosic matrix” or “medicated cellulosic matrix” or “cellulosic matrix” is intended to encompass any type of cellulose produced via fermentation or synthesized by a bacteria such as but not limited to, Gluconacetobacter xylinus, Gluconacetobacter hansenii, Acetobacter xylinus, Acetobacter xylinum, or mutants or genetic variants thereof. Microbial celluloses are normally available in a gel produced in a bacterial, fungal or algal culture.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) cellulose and its derivatives, such as sodium carboxymethyl cellulose, ethyl cellulose and cellulose acetate; (2) starches, such as corn starch and potato starch; (3) sugars, such as lactose, glucose and sucrose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. The term may also include preservatives.

Before the compositions, methods and delivery systems of the present disclosure are described in greater detail, it is to be understood that the invention is not limited to particular embodiments and may vary. It is also to be understood that the terminology used herein is for the purpose of describing particular embodiments only and is not intended to be limiting.

### Preparation of Synergistic Essential Oil Compositions

Essential oils or active ingredients having anti-fungal activity and antibacterial activity were chosen for preparation of the oil compositions.

In one embodiment, the essential oil or active ingredient is extracted from a plant belonging to a member of the Lamiaceae family selected from a group comprising Thymus, Ocimum, Origanum, Monarda and the like.

In another embodiment, the essential oil is Thymus vulgaris essential oil or thymol essential oil.

In yet another embodiment, the active ingredient present is Thymol (2-isopropyl-5-methylphenol or IPMP).

In another embodiment, the essential oil or active ingredient is extracted from a plant selected from a group comprising clove (Syzygium aromaticum), cinnamon (Cinnamomum verum), bay leaf (Cinnamomum tamala, Umbellularia californica, Laurus nobilis, Pimenta racemose and Litsea glaucescens), tulsi (Ocimum tenuiflorum), nutmeg (Myristic sp.), pepper (Piper nigrum) and the like.

In another embodiment, the essential oil is a eugenol essential oil.

In yet another embodiment, the active ingredient is eugenol (2-Methoxy-4-(prop-2-en-1-yl) phenol).

In one embodiment, the essential oil or active ingredient is extracted from a plant belonging to a member of the Origanum family selected from a group comprising Origanum vulgare, Origanum compactum, Origanum dictamnus, Origanum microphyllum, Origanum glanulosus, Origanum onites, Origanum scabrum and the like.

In yet another embodiment, the essential oil or active ingredient is extracted from a plant belonging to a member of the Labiatae family of plants selected from a group comprising basil (Ocimum basilicum), mint (Mentha sp.), rosemary (Rosmarinus officinalis), sage (Salvia officinalis), savory (Satureja sp.), marjoram (Origanum majorana), hyssop (Hyssopus officinalis), lavender (Lavandula sp.) and the like.

In another embodiment, the essential oil is carvacrol essential oil.

In yet another embodiment, the active ingredient present is carvacrol (5-Isopropyl-2-methylphenol).

In another embodiment, the essential oil composition comprises one or more oils and/or active ingredients as described above.

In yet another embodiment, the essential oil composition comprises two or more oils and/or active ingredients as described above.

In another embodiment, the essential oils are present at a volume/volume ratio from 5:1 to 1:5, or 5:1 to 1:4, or 5:1 to 1:3, or 5:1 to 1:2, or 5:1 to 1:1.

In yet another embodiment, the essential oil composition comprises three or more oils and/or active ingredients as described above.

In another embodiment, each essential oils is present in the composition at a volume/volume ratio from 10:1 to 1:10, or 10:1 to 1:9, or 10:1 to 1:8, or 10:1 to 1:7, 10:1 to 1:6, or 10:1 to 1:5, or 10:1 to 1:4, or 10:1 to 1:3, or 10:1 to 1:2, or 10:1 to 1:1.

In another embodiment, each essential oil is present in the essential oil composition at a concentration range of about 5-10% v/v, about 10-15% v/v, about 15-20% v/v, about 20-25% v/v, about 25-30% v/v, 30-35% v/v, about 35-40% v/v, about 40-45% v/v, about 45-50% v/v, 50-55% v/v, about 55-60% v/v, about 60-65% v/v, about 65-70% v/v, 70-75% v/v, about 75-80% v/v, about 80-85% v/v, about 85-90% v/v, about 90-95% v/v.

In a further embodiment, each essential oil is present in the composition comprises four or more oils and/or active ingredients as described above.

The essential oil composition of the present invention may further comprise a pharmaceutically acceptable carrier, excipient or preservatives. The carriers include but are not limited to, solid diluents or fillers, excipients, sterile aqueous media and various non-toxic organic solvents. Dosage unit forms or pharmaceutical compositions include tablets, capsules, pills, powders, granules, aqueous and non-aqueous oral solutions and suspensions, creams, hard candies, lozenges, troches, sprays, salves, suppositories, gels, pastes, ointments, jellies, lotions, injectable solutions, elixirs, syrups, and parenteral solutions packaged in containers adapted for subdivision into individual doses.

### Encapsulation of the Essential Oil in Microcapsules

Microencapsulation is a method in which tiny particles or droplets are surrounded by a coating wall, or are embedded in a homogeneous or heterogeneous matrix, to form small capsules.

Microcapsules enable protection and assist the controlled/sustained release of essential oils or active ingredient over a certain period of time.

Embodiments of the present invention cover core-shell microcapsules for essential oil or active ingredient delivery. The polymeric wall of the microcapsules works as a permeable element with a selectivity that can determine the release behavior of the core material.

Permeation enhancers and biocompatible surfactants are used in the microcapsule formulation in order to increase the diffusion and enhance the overall absorptivity of essential oils or active ingredient into the oral mucosa.

In one embodiment, the essential oil microcapsule formulation can be varied according to the site of the application.

In another embodiment, essential oil microcapsules are prepared by a modified coacervation-phase separation method.

In another embodiment, process parameters such as homogenization time, temperature and spray drying speeds were modified.

In a further embodiment, the homogenization time in the coacervation-phase separation method is in the range of 15 sec to 30 minutes.

In a further embodiment, the temperature in the coacervation-phase separation method is in the range of −20 to 50° C.

In a non-limiting embodiment, the capsule shell material is selected from a group comprising chitosan, polylactide (PLA), Polymethyl methacrylate (PMMA), poly(N-isopropylmethacrylamide) (PNIPAM) and alginates.

In another embodiment, the concentration of the material in a capsule shell is in the range of 0.2 to 10% (w/v).

In another embodiment, the capsule shell is configured for immediate pH-responsive release.

In yet another embodiment, the capsule shell is configured for adhesion to the mucosal lining of the cavity, followed by erosion of the capsule shell for release of the essential oils or active ingredients.

In another embodiment, the modified coacervation-phase separation method comprises the steps of:
a. preparing an emulsion comprising essential oil composition as described herein, a surfactant and water; and
b. adding a polymer solution and a cross-linker to the emulsion to obtain the microcapsule composition.

Non-limiting examples of surfactants suitable in embodiments of the present invention are tweens (such as Tween 20, Tween 80 and the like), spans, poloxamer and the like.

In one embodiment, the mode of cross-linking is ionic cross-linking or physicochemical cross-linking.

In a non-limiting embodiment, polymer solution forms the capsule shell of the microcapsules. Accordingly, polymer solution material is selected from a group comprising chitosan, polylactide (PLA), Polymethyl methacrylate (PMMA), poly(N-isopropylmethacrylamide) (PNIPAM) and alginates.

Non-limiting examples of cross-linkers suitable in embodiments of the present invention are TPP (Sodium Tripolyphosphate), NaOH, OCMTS (octamethylcyclotetrasiloxane), glutaraldehyde, genipin, and the like.

In another embodiment, for preparation of an emulsion, essential oil composition was homogenized with water and Poloxamer 188 as a surfactant.

In another embodiment, the surfactant is present at a concentration in the range from 1.5 wt % to 7.5 wt % in the emulsion.

In a further embodiment, polylactide (Polylactic acid) was used as a polymer for coating the water-oil emulsion.

In another embodiment, the polymer concentration is present at a concentration in the range from 2.5 wt % to 5.0 wt % in the emulsion.

In a further embodiment, Octamethylcyclotetrasiloxane (OCMTS) was added as a cross-linker for preparing the microcapsules.

In a further embodiment, Pluronic f68 was added to the mixture during washing to prevent capsule deformation.

In another embodiment, the microcapsules are purified using techniques known in the art.

### Preparation of Medicated Cellulosic Matrices

For further enhancement of the efficacy of the therapeutic modalities, microcapsules of essential oils embedded in a matrix.

Amongst various matrices, biocompatible hydrogels with high specific surface area, high compressive strength and loading capacity are preferable. In light of these requirements, microbial cellulose is preferable due to its nanofibrous and microporous nature. Microbial cellulose is produced by Gluconacetobacter xylinus or Gluconacetobacter hansenii or Acetobacter xylinus bacteria as a dilute hydrogel of pure semicrystalline cellulose nanofibers with high porosity and water holding capacity along with an additional advantage of in situ manipulability.

The microbial cellulose used for the composite could also be obtained from fruit juices, coconut water, tea, etc. The properties of microbial cellulose such as crystallinity, porosity, mechanical strength, absorbing capacity, swellability, gaseous and water vapor transmission rate could be altered according to the site of usage by modifiers (during and/or post-synthesis) and by altering the drying processes (Freeze-drying and oven drying). In situ modifications could also be done during microbial cellulose production using modifiers such as polyethylene glycol (2000-20000 M.W) PEG, carboxymethyl cellulose (5000 to 20000 M.W) CMC. Further agents such as nalidixic acid can be used for in situ modification to alter porosity, fibril dimension, pore size, strength and crystallinity. Surface modification of microbial cellulose can also be done with Chitosan or PNIPAM (Poly(N-isopropylacrylamide) for pH and thermosensitive release.

In one aspect, the invention provides for preparation of monolithic mononuclear or polynuclear core-shell microcapsules encapsulated with three different oils or active ingredients separately within the same capsule and the embedding the microcapsules in microbial cellulosic matrix.

In one embodiment the delivery system for essential oil or active ingredient microcapsules is a cellulosic matrix delivery system.

In another embodiment, the cellulosic matrix is a conventionally used natural or a synthetic matrix.

In one embodiment, the matrix is a biocompatible hydrogel with high specific surface area, high compressive strength and loading capacity.

In another embodiment, the cellulosic matrix is microbial cellulose.

In another embodiment, the cellulosic matrix is derived from a bacterial or fungal source.

In another embodiment, the cellulose producing bacterial culture is Gluconacetobacter xylinus or Gluconacetobacter hansenii or mutants or genetic variants thereof.

In another embodiment, the cellulose producing bacterial culture is Acetobacter xylinus, Acetobacter xylinum or mutants or genetic variants thereof.

In another embodiment, the Na to de coco is used as a cellulosic matrix.

In yet another embodiment, the matrix is modified with carboxymethyl cellulose (CMC) or polyethylene glycol (PEG).

In another embodiment, the pH of the matrix is in the range from 2 to 9. Preferably, the pH is in the range from 3 to 5.

In another embodiment, the essential oil or active ingredient microcapsules are incubated with freeze-dried microbial cellulosic matrix for a period in the range of 20 mins-30 hrs, as per the application.

In one embodiment, the cellulosic matrix optionally comprises suitable excipients.

In another embodiment of the invention, the excipients in the cellulosic matrix are selected from the group consisting of flavors, plasticizers, dry-binders, tableting aids, anti-caking agents, emulsifiers, antioxidants, enhancers, surfactants, cross-linkers, absorption enhancers, sweeteners, softeners, coloring agents, active ingredients, water-soluble indigestible polysaccharides, water-insoluble polysaccharides and any combination thereof.

Non-limiting examples of flavors or flavoring agents suitable in embodiments of the present invention are peppermint, spearmint, menthol, eucalyptus, clove oil, bay oil, anise, thyme, cedar leaf oil, nutmeg oil, coconut, coffee, chocolate, vanilla, grape fruit, orange, lime, menthol, caramel, honey, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, plum essence and the like.

In the embodiments of the present invention, sweeteners include, but are not limited to glucose, sucrose, sucralose, aspartame, salts of acesulfame, alitame, saccharin and its salts, cyclamic acid and its salts, glycyrrhizin, dihydrochalcones, thaumatin, monellin, stevioside and the like, alone or in combination.

Any regulatory approved excipient may be used to enhance the properties of the cellulosic matrix.

In a further embodiment, the concentration of excipients is in the range of 0.1 to 20% (w/v).

In another embodiment, the cellulosic matrix of the present invention comprises embedded monolithic polynuclear core-shell microcapsules encapsulated with one or more essential oils or active ingredients.

In yet another embodiment, the retention time and duration of drug release is upto a period of 2-12 hrs.

In another embodiment, the thickness, size and properties of the cellulosic matrix can be modified to enhance the absorption properties.

In yet another embodiment, the cellulosic matrix can absorb a minimum of 2 mL of exudates for oral application. In a preferred embodiment, the cellulosic matrix can absorb a minimum of 5 mL of exudates for vaginal applications.

### Micronized Spray

The freeze-dried essential oil or active ingredient microcapsules of the present invention have potential applications as a micronized spray for inhalation through nebulizer/rotahaler to treat respiratory tract infections, such as tuberculosis, pneumonia and the like.

### EXAMPLES

The invention will now be further illustrated by the following non-limiting examples. The specific examples below are to be construed as merely illustrative, and not limitative of the remainder of the disclosure in any way whatsoever. The components and/or reagents of the present disclosure are commercially available and/or can be prepared according to methods readily available to a skilled person.

### Example 1: Preparation of Oil Compositions and Synergistic Effects of the Oil Compositions

Three essential oils, thymol oil, eugenol oil and carvacrol oil (obtained from Sigma Aldrich, USA) were chosen for preparation of oil compositions.

Different two-oil and three-oil compositions with varying ratios were prepared in order to check the performance of the oils for inhibition of Candida albicans. Fluconazole and saline were used as positive and negative controls, respectively.

The different two-oil and three-oil compositions were compared with the controls in a large number of experiments performed as demonstrated below:

TABLE 1
Synergistic effects of the oil compositions

| | | | | Time | |
|---|---|---|---|---|---|
| | | | Inhibition | period | |
| | | | zone | (in | |
| Oil Composition | Ratio | Volume | diameter | hrs) | FIG. |
| Fluconazole | Positive | 200 μg | 5.3 | 24 | FIG. |
| | Control | | | | 4(a) |
| | | | 4.5 | 48 | FIG. |
| | | | | | 4(b) |
| | | | 3.4 | 72 | FIG. |
| | | | | | 4(c) |
| Saline | Negative | 100 μL | 0 | 24 | FIG. |
| | Control | | | | 4(a) |
| | | | 0 | 48 | FIG. |
| | | | | | 4(b) |
| | | | 0 | 72 | FIG. |
| | | | | | 4(c) |
| Thymol | — | 50 μL | 2.5 | 24 | FIG. |
| | | | | | 1(a) |
| | | 100 μL | 3.1 | 24 | FIG. |
| | | | | | 1(a) |
| | | 150 μL | 3.9 | 24 | FIG. |
| | | | | | 1(a) |
| | | 200 μL | 4.2 | 24 | FIG. |
| | | | | | 1(a) |
| Eugenol | — | 50 μL | 2.0 | 24 | FIG. |
| | | | | | 1(b) |
| | | 100 μL | 3.5 | 24 | FIG. |
| | | | | | 1(b) |
| | | 150 μL | 4.1 | 24 | FIG. |
| | | | | | 1(b) |
| | | 200 μL | 4.3 | 24 | FIG. |
| | | | | | 1(b) |
| Carvacrol | — | 50 μL | 3.2 | 24 | FIG. |
| | | | | | 1(c) |
| | | 100 μL | 3.8 | 24 | FIG. |
| | | | | | 1(c) |
| | | 150 μL | 4.5 | 24 | FIG. |
| | | | | | 1(c) |
| | | 200 μL | 5.1 | 24 | FIG. |
| | | | | | 1(c) |
| Thymol:Carvacrol | 1:1 | 100 μL | 5.8 | 24 | FIG. |
| | | | | | 1(d) |
| | | 150 μL | 6.0 | 24 | FIG. |
| | | | | | 1(e) |
| | | 200 μL | 6.2 | 24 | FIG. |
| | | | | | 1(f) |
| | 1:2 | 50 μL | 4.0 | 48 | FIG. |
| | | | | | 2(a) |
| | | | | | (Zone |
| | | | | | A1- |
| | | | | | 50) |
| | | 50 μL | 3.8 | 72 | FIG. |
| | | | | | 2(b) |
| | | | | | (Zone |
| | | | | | A1- |
| | | | | | 50) |
| | | 100 μL | 6.2 | 48 | FIG. |
| | | | | | 2(a) |
| | | | | | (Zone |
| | | | | | A1- |
| | | | | | 100) |
| | | 100 μL | 5.8 | 72 | FIG. |
| | | | | | 2(b) |
| | | | | | (Zone |
| | | | | | A1- |
| | | | | | 100) |
| | 2:1 | 50 μL | 4.1 | 48 | FIG. |
| | | | | | 2(a) |
| | | | | | (Zone |
| | | | | | A2- |
| | | | | | 50) |
| | | 50 μL | 3.7 | 72 | FIG. |
| | | | | | 2(b) |
| | | | | | (Zone |
| | | | | | A2- |
| | | | | | 50) |
| | | 100 μL | 6.2 | 48 | FIG. |
| | | | | | 2(a) |
| | | | | | (Zone |
| | | | | | A2- |
| | | | | | 100) |
| | | 100 μL | 5.9 | 72 | FIG. |
| | | | | | 2(b) |
| | | | | | (Zone |
| | | | | | A2- |
| | | | | | 100) |
| Thymol:Eugenol | 1:1 | 100 μL | 5.3 | 24 | FIG. |
| | | | | | 1(d) |
| | | 150 μL | 5.4 | 24 | FIG. |
| | | | | | 1(e) |
| | | 200 μL | 6.0 | 24 | FIG. |
| | | | | | 1(f) |
| | 1:2 | 50 μL | 3.5 | 48 | FIG. |
| | | | | | 2(a) |
| | | | | | (Zone |
| | | | | | A3- |
| | | | | | 50) |
| | | 50 μL | 3.0 | 72 | FIG. |
| | | | | | 2(b) |
| | | | | | (Zone |
| | | | | | A3- |
| | | | | | 50) |
| | | 100 μL | 6.5 | 48 | FIG. |
| | | | | | 2(a) |
| | | | | | (Zone |
| | | | | | A3- |
| | | | | | 100) |
| | | 100 μL | 5.9 | 72 | FIG. |
| | | | | | 2(b) |
| | | | | | (Zone |
| | | | | | A3- |
| | | | | | 100) |
| | 2:1 | 50 μL | 3.4 | 48 | FIG. |
| | | | | | 2(a) |
| | | | | | (Zone |
| | | | | | A4- |
| | | | | | 50) |
| | | 50 μL | 2.9 | 72 | FIG. |
| | | | | | 2(b) |
| | | | | | (Zone |
| | | | | | A4- |
| | | | | | 50) |
| | | 100 μL | 6.5 | 48 | FIG. |
| | | | | | 2(a) |
| | | | | | (Zone |
| | | | | | A4- |
| | | | | | 100) |
| | | 100 μL | 5.8 | 72 | FIG. |
| | | | | | 2(b) |
| | | | | | (Zone |
| | | | | | A4- |
| | | | | | 100) |
| Eugenol:Carvacrol | 1:1 | 100 μL | 5.7 | 24 | FIG. |
| | | | | | 1(d) |
| | | 150 μL | 5.8 | 24 | FIG. |
| | | | | | 1(e) |
| | | 200 μL | 5.9 | 24 | FIG. |
| | | | | | 1(f) |
| | 1:2 | 50 μL | 2.7 | 48 | FIG. |
| | | | | | 2(a) |
| | | | | | (Zone |
| | | | | | A5- |
| | | | | | 50) |
| | | 50 μL | 2.2 | 72 | FIG. |
| | | | | | 2(b) |
| | | | | | (Zone |
| | | | | | A5- |
| | | | | | 50) |
| | | 100 μL | 6.1 | 48 | FIG. |
| | | | | | 2(a) |
| | | | | | (Zone |
| | | | | | A5- |
| | | | | | 100) |
| | | 100 μL | 5.7 | 72 | FIG. |
| | | | | | 2(b) |
| | | | | | (Zone |
| | | | | | A5- |
| | | | | | 100) |
| | 2:1 | 50 μL | 2.3 | 48 | FIG. |
| | | | | | 2(a) |
| | | | | | (Zone |
| | | | | | A6- |
| | | | | | 50) |
| | | 50 μL | 2.0 | 72 | FIG. |
| | | | | | 2(b) |
| | | | | | (Zone |
| | | | | | A6- |
| | | | | | 50) |
| | | 100 μL | 5.6 | 48 | FIG. |
| | | | | | 2(a) |
| | | | | | (Zone |
| | | | | | A6- |
| | | | | | 100) |
| | | 100 μL | 4.8 | 72 | FIG. |
| | | | | | 2(b) |
| | | | | | (Zone |
| | | | | | A6- |
| | | | | | 100) |
| Thymol:Eugenol:Carvacrol | 1:1:1 | 100 μL | 6.0 | 24 | FIG. |
| | | | | | 1(d) |
| | | 150 μL | 6.4 | 24 | FIG. |
| | | | | | 1(e) |
| | | 200 μL | 6.5 | 24 | FIG. |
| | | | | | 1(f) |
| | 1:2:3 | 50 μL | 6.2 | 48 | FIG. |
| | | | | | 3(a) |
| | | | | | (Zone |
| | | | | | B1) |
| | | 50 μL | 5.1 | 72 | FIG. |
| | | | | | 3(b) |
| | | | | | (Zone |
| | | | | | B1) |
| | 3:2:1 | 50 μL | 6.0 | 48 | FIG. |
| | | | | | 3(a) |
| | | | | | (Zone |
| | | | | | B2) |
| | | 50 μL | 4.8 | 72 | FIG. |
| | | | | | 3(b) |
| | | | | | (Zone |
| | | | | | B2) |
| | 2:3:1 | 50 μL | 3.6 | 48 | FIG. |
| | | | | | 3(a) |
| | | | | | (Zone |
| | | | | | B3) |
| | | 50 μL | 3.0 | 72 | FIG. |
| | | | | | 3(b) |
| | | | | | (Zone |
| | | | | | B3) |
| | 3:1:2 | 50 μL | 3.5 | 48 | FIG. |
| | | | | | 3(a) |
| | | | | | (Zone |
| | | | | | B4) |
| | | 50 μL | 2.9 | 72 | FIG. |
| | | | | | 3(b) |
| | | | | | (Zone |
| | | | | | B4) |
| | 2:1:3 | 50 μL | 3.4 | 48 | FIG. |
| | | | | | 3(a) |
| | | | | | (Zone |
| | | | | | B5) |
| | | 50 μL | 2.8 | 72 | FIG. |
| | | | | | 3(b) |
| | | | | | (Zone |
| | | | | | B5) |
| | 1:3:2 | 50 μL | 5.8 | 48 | FIG. |
| | | | | | 3(a) |
| | | | | | (Zone |
| | | | | | B6) |
| | | 50 μL | 4.5 | 72 | FIG. |
| | | | | | 3(b) |
| | | | | | (Zone |
| | | | | | B6) |

Table 1 exhibits that the combination of thymol oil, eugenol oil and carvacrol oil in varying ratios exhibit synergistic effect for inhibition of Candida albicans and can be used for prevention or treatment of candidiasis.

### Example 2: Encapsulation of the Essential Oil in Microcapsules

Essential oil microcapsules (herein, also referred to as oil colloids) were prepared by coacervation-phase separation method. Encapsulation of the oil compositions in microcapsules was achieved in three stages:

Stage 1: Emulsification

An emulsion was prepared by homogenizing the oil composition with water and Poloxamer 188 surfactant at a 0.3 v/v %. The surfactant reaction mixture of Poloxamer 188 was prepared at different concentrations (1.5 wt %, 2.5 wt %, 5.0 wt % and 7.5 wt %) with Tween 20 and deionized water. Briefly, 250 mL of surfactant of varying concentration was used for 75 mL of oil. The oil composition was added dropwise, and the surfactant reaction mixture was homogenized using a homogenizer at 15000 rpm for first 5 minutes followed by 11000 rpm for 90 seconds.

Stage 2: Coating of Core Material

250 mL of emulsion prepared in Stage 1 was stirred at 1000 rpm at room temperature for 1 hour. The polymer used for coating was polylactide (Polylactic acid). Different polylactide solutions were prepared in dimethylformamide at 1.5 wt % and 3.0 wt %. Polylactide solutions were added dropwise to the oil-water emulsion while being stirred at 200 rpm at room temperature for 3 hours.

Stage 3: Hardening/Cross-Linking

After coating, Octamethylcyclotetrasiloxane (OCMTS) was added as a cross-linker and the reaction mixture was allowed to stand for 2 hours at room temperature for cross-linking. The reaction mixture was subjected to phase separation by decantation, followed by washing with 30% v/v ethanol and n-hexane. Pluronic f68 was added to the mixture during washing to prevent capsule deformation. The oil capsules were purified using a 0.2 μm syringe filter followed by gas chromatography.

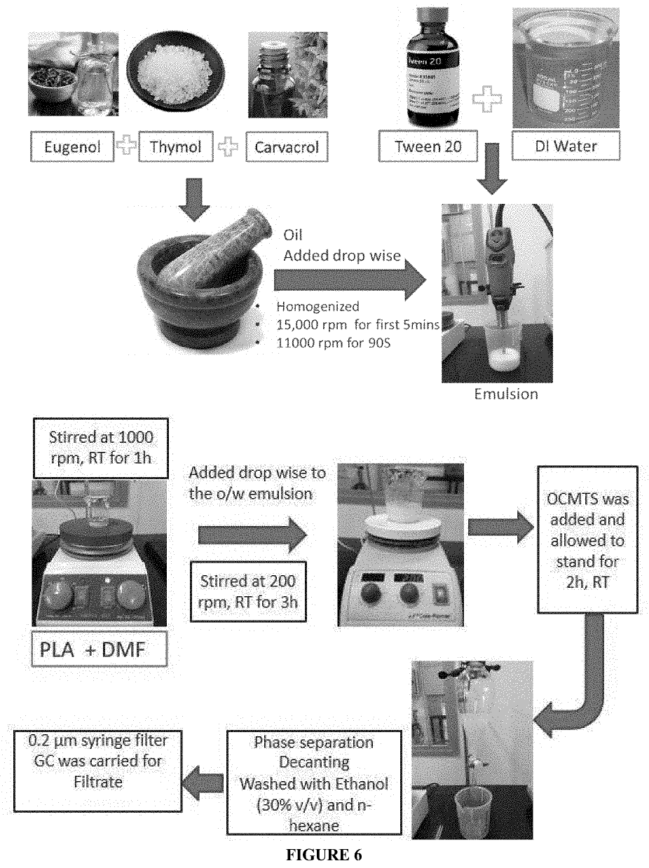

The encapsulation process is schematically depicted in FIG. 5 and FIG. 6.

### Example 3: Characterization and Size Determination of the Microcapsules by Microscopic Studies

The microcapsules were characterized using various microscopic studies.

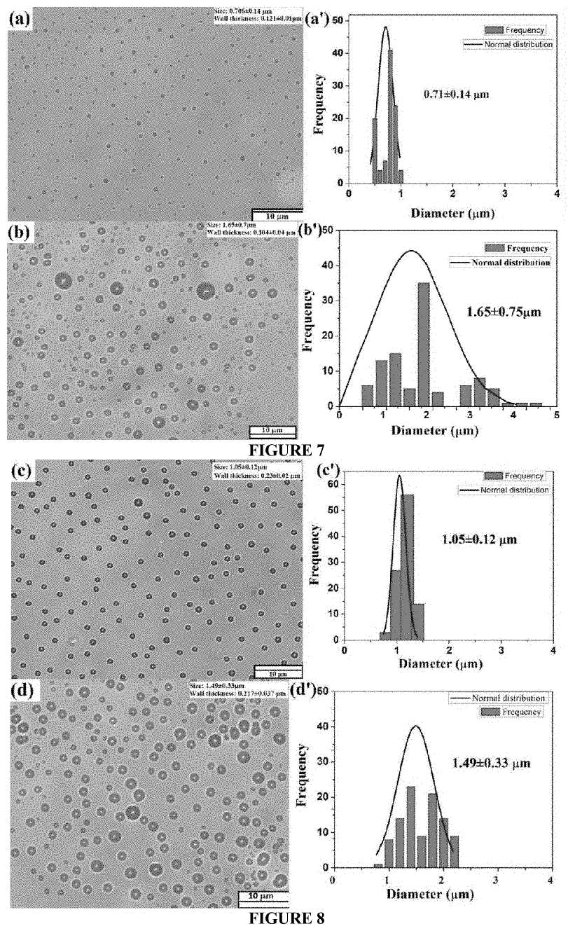

Optical microscopic studies were performed to check the encapsulation efficiency and determining the diameter. The effect of varying the polymer and surfactant was studied on microcapsules. Essentially, it was seen that a higher surfactant or higher polymer concentration resulted in more disperse and smaller microcapsules. Further, higher surfactant or polymer concentration led to clear boundaries which indicate stable dispersion. On the other hand, lower surfactant or polymer concentration led to polydisperse microcapsules and unstable dispersion. The results of optical microscopy are indicated in FIG. 7 and FIG. 8.

FIG. 7 depicts the optical microstructure of PLA_{1.5}Pol_{5.0} and PLA_{1.5}Pol_{2.5}. FIG. 8 depicts the optical microstructure of PLA_{3.0}Pol_{5.0} and PLA_{3.0}Pol_{2.5}.

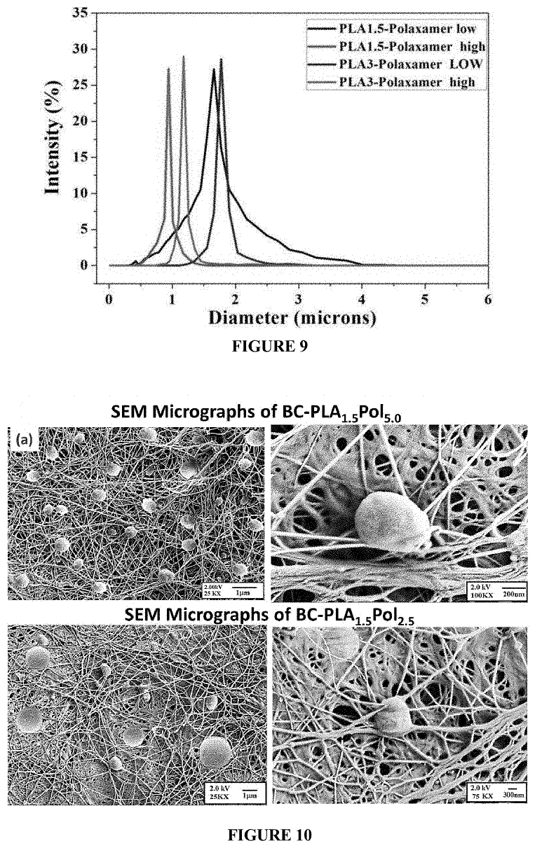

Dynamic Light Scattering Microscopy (DLS) performed on the microcapsules to determine the polydispersity index (PDI) and the diameter. The DLS microscopic studies exhibited that least size and narrow size distribution was observed for microcapsules synthesized with high surfactant irrespective of polymer concentration. Further, larger-sized capsules with high PDI was obtained for microcapsules synthesized with low surfactant and low polymer. The results of the DLS microscopic studies are exhibited in FIG. 9.

The diameter of the microcapsules was also measured using Scanning Electron Microscopy (SEM).

The results are provided in the below table.

TABLE 2
Diameter and PDI measurement of the microcapsules

| | Size of microcapsules (μm) | | | | |
|---|---|---|---|---|---|
| | | | | Optical | |
| | | DLS | | Microscopy | SEM |
| Sr. | | Diameter | | Diameter | Diameter |
| No. | Sample | (μm) | PDI | (μm) | (μm) |
| (a) | PLA_{1.5}Pol_{5.0} | 0.9 | 0.219 | 1.1 ± 0.2 | 0.6 ± 0.1 |
| (b) | PLA_{1.5}Pol_{2.5} | 1.8 | 0.726 | 1.8 ± 0.7 | 1.5 ± 0.5 |
| (c) | PLA_{3.0}Pol_{5.0} | 1.2 | 0.228 | 1.2 ± 0.1 | 1.0 ± 0.1 |
| (d) | PLA_{3.0}Pol_{2.5} | 1.6 | 0.301 | 1.4 ± 0.3 | 1.3 ± 0.2 |

### Example 5: Calculation of Encapsulation Efficiency

The obtained oil microcapsules were characterized by studying the optical microstructure as provided in Example 3. The size of the microcapsules was determined as provided in Table 2. The encapsulation efficiency (EE %) was also calculated based on the formula:

Encapsulation Efficiency (EE %)=(mₜₒₜₐₗ−mₒᵤₜ/mₜₒₜₐₗ)*100, wherein mote represents the amount of loaded core material and mₒᵤₜ represents the amount of non-encapsulated model core material. Four illustrative representations are provided below:

TABLE 3
Calculation of Encapsulation Efficiency

| | | | | Encapsulation |
|---|---|---|---|---|
| | | mₜₒₜₐₗ | m_{encapsulated} | Efficiency |
| Concentration | | (mg) | (mg) | (EE %) |
| PLA 1.5% (w/w) | Poloxamer 2.5 | 750 | 497.64 | 66.5 |
| | Poloxamer 5.0 | 750 | 586.29 | 78.2 |
| PLA 3.0% (w/w) | Poloxamer 2.5 | 750 | 575.51 | 76.7 |
| | Poloxamer 5.0 | 750 | 636.42 | 84.8 |

The below table provides the encapsulation efficiency of the microcapsules formed with varying amount of polymer and surfactant.

TABLE 4
Encapsulation Efficiency (EE %)
Formulations

| | Poloxamer | Encapsulation | Size |
|---|---|---|---|
| PLA | 188 | Efficiency (EE %) | (in μm) |
| 1.5 | 1.5 | 54.2 | 2.5 ± 1.2 |
| | 2.5 | 66.5 | 1.8 ± 0.7 |
| | 5.0 | 78.2 | 1.1 ± 0.2 |
| | 7.5 | 78.3 | 1.1 ± 0.1 |
| 3.0 | 1.5 | 65.5 | 1.5 ± 0.9 |
| | 2.5 | 76.7 | 1.4 ± 0.3 |
| | 5.0 | 84.8 | 1.2 ± 0.1 |
| | 7.5 | 85.1 | 1.2 ± 0.1 |

### Example 6: Preparation of Microbial Cellulosic Matrix Loaded with Oil Microcapsules

Microcapsules of essential oils prepared in Example 2 were embedded in a microbial cellulosic matrix. Microbial cellulose produced by Gluconacetobacter xylinus, Gluconacetobacter hansenii or Acetobacter xylinus were used for the purposes of the invention.

The microcapsules of the essential oil were incubated with freeze-dried microbial cellulosic matrix for a period in the range of 20 mins-30 hrs to obtain microbial cellulosic matrix loaded with oil microcapsules.

Freeze-dried microbial cellulose pellicles of 13 cm diameter and 0.5 mm thickness which weigh 60 mg were taken to prepare composites. The bacterial cellulose pellicles were soaked in 15 ml of microcapsules which corresponds to 1.5 mg of total oil content, for 4-6 hrs and then freeze-dried.

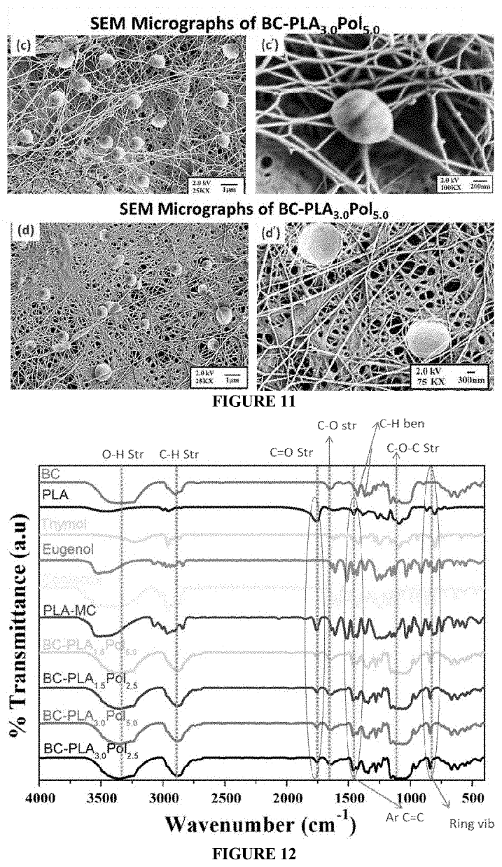

Scanning Electron Microscopic studies were conducted to check the embedding of the oil microcapsules in the microbial cellulosic matrix. The results of the studies are depicted in FIG. 10 and FIG. 11.

The studies confirmed that the oil microcapsule loaded cellulosic matrix confers effective and advantageous effects.

### Example 7: FTIR Spectroscopic Studies

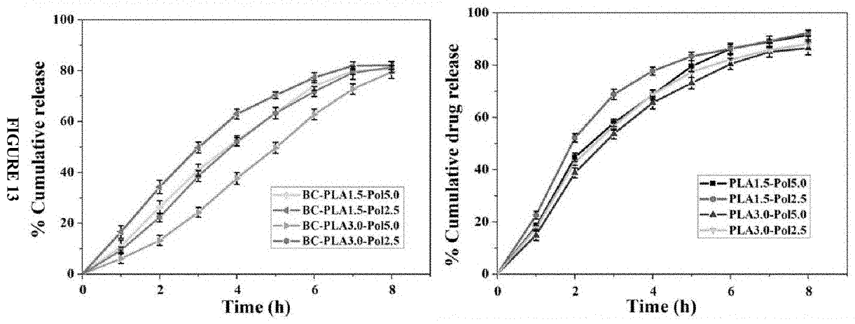

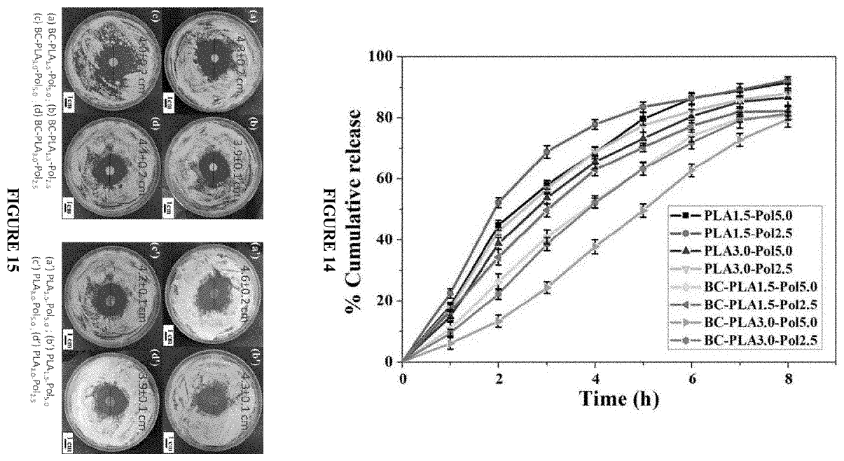

An oil microcapsule (prepared with thymol oil, eugenol oil and carvacrol oil) loaded cellulosic matrices was characterized by FTIR for demonstrating chemical compatibility. The FTIR studies are depicted in FIG. 12.

The FTIR studies show that there is just a minor shift in the wavenumber and there are no chemical modifications. The vibrations at 1750 cm⁻¹, 1500 cm⁻¹ and 750 cm⁻¹ indicate the presence of all the three-oil microcapsule in the cellulosic matrix.

### Example 8: In Vitro Drug Release Studies in Simulant Vaginal Fluid (SVF)

In vitro drug release studies were conducted in simulant vaginal fluid for different oil microcapsules and cellulosic matrices loaded with oil microcapsules. Cumulative drug release was plotted against time. The studies are depicted in FIGS. 13 and 14.

Simulated vaginal fluid (SVF) of pH ˜4.5 was used for the release studies to match the pH of vaginal mucosa. The composition of SVF is provided below. All the chemicals used for the preparation were purchased from Sigma Aldrich and taken in quantities mention in the below table.

| Compound | Weight (g)/L |
|---|---|
| NaCl | 3.51 |
| KOH | 1.40 |
| Ca(OH)₂ | 0.222 |
| BSA | 0.018 |
| Lactic acid | 2.0 |
| Acetic acid | 1.0 |
| Glycerol | 0.16 |
| Urea | 0.4 |
| Glucose | 5.0 |
| 0.1N HCl | pH~4.5 |

The studies indicate that the oil microcapsules exhibit burst release at the range of 35-52% in the first 2 hours and show a linear increment release for 2-6 hours, followed by a plateau phase from 6-8 hours.

The loaded cellulosic matrices exhibited burst release at the range of 13-34% in the first 2 hours and show a linear increment release for 2-6 hours, slow release from 6-8 hours, followed by a plateau phase after 8 hours.

The studies indicate that the microcapsules are suited for burst release applications, while the cellulosic matrices are suited for sustained release applications.

### Example 9: Mechanism of Release

In order to understand the mode of release in the microcapsules and cellulosic matrices. To understand the dissolution mechanisms from the matrices and microcapsules, the release data were fitted into the first-order model and Korsmeyer peppas model (Dash, S. et. al. 2010).

TABLE 5
Release mechanism of microcapsules from cellulosic matrices

| | | BC- | BC- | BC- | BC- |
|---|---|---|---|---|---|
| | | PLA_{3.0}- | PLA_{3.0}- | PLA_{1.5}- | PLA_{1.5}- |
| Model | | Pol_{5.0} | Pol_{2.5} | Pol_{5.0} | Pol_{2.5} |
| First order | | 0.9051 | 0.96174 | 0.96965 | 0.96174 |
| Korsmeyer | correlation | 0.9968 | 0.995 | 0.99326 | 0.9167 |
| peppas | coefficient | | | | |
| | (R2) | | | | |
| | n | 0.71 | 0.47 | 0.88 | 0.75 |

The n value is used to characterize different release mechanisms. The n value for the matrices lies in the range of 0.45 to 0.89 which indicates a non-Fickian transport.

TABLE 6
Release mechanism of microcapsules from cellulosic matrices

| | PLA_{3.0}- | PLA_{3.0}- | PLA_{1.5}- | PLA_{1.5}- |
|---|---|---|---|---|
| Model | Pol_{5.0} | Pol_{2.5} | Pol_{5.0} | Pol_{2.5} |
| First order | 0.99473 | 0.9935 | 0.99189 | 0.99495 |
| Korsmeyer peppas correlation | 0.92864 | 0.8774 | 0.92184 | 0.95943 |
| coefficient (R2) | | | | |

Based on the studies, it is can be said that the release of oils from the cellulosic matrices follows diffusion-controlled release system or matrix swelling controlled release system.

N value close to 1 in Korsmeyer peppas model indicated that the release is zero order. Amongst all the samples tested, n value of composites (BC-PLA_{3.0}-Pol_{5.0}, BC-PLA_{L5}-Pol_{5.0} and BC-PLA_{3.0}-Pol_{2.5}) is closer to 1. Hence, it can be inferred that the release system is close to zero-order release system.

### Example 11: Antifungal Activity of Microcapsules and Bacterial Cellulose Loaded Microcapsules

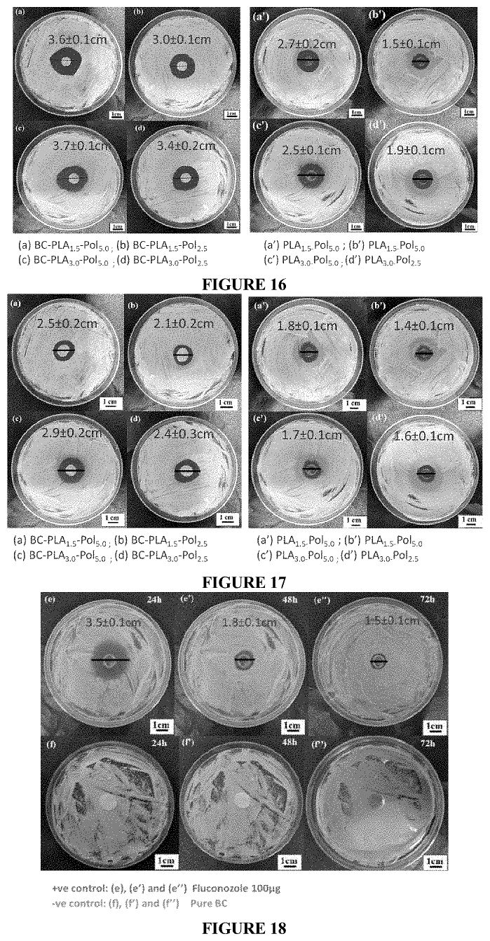

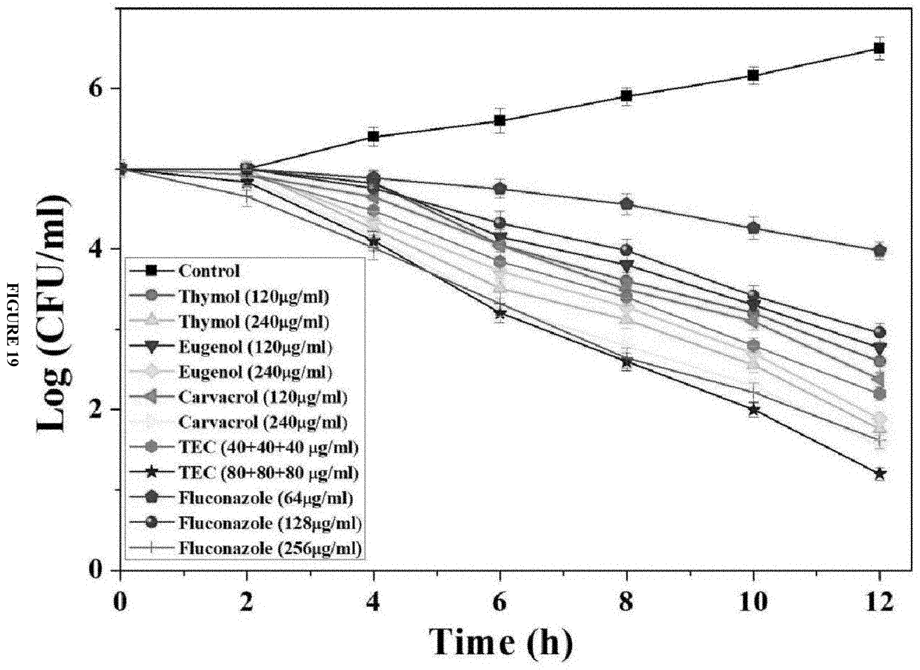

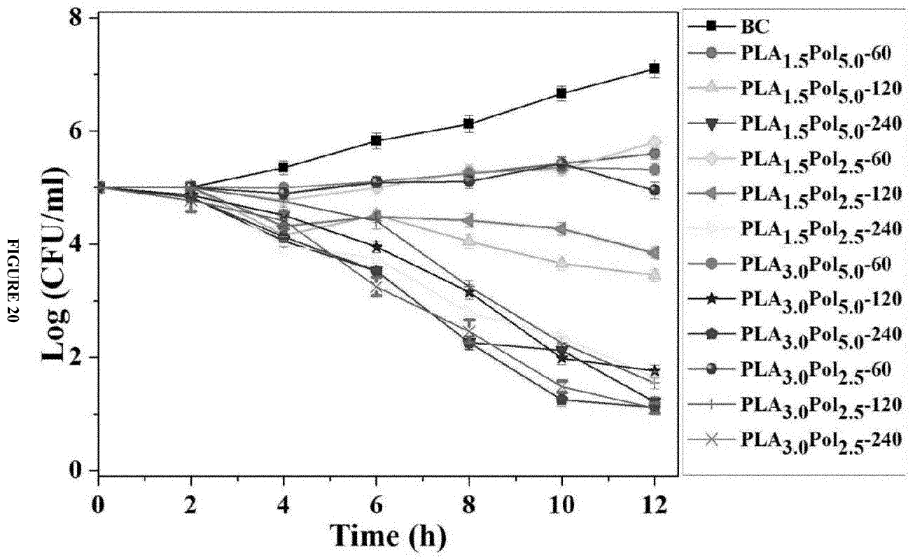

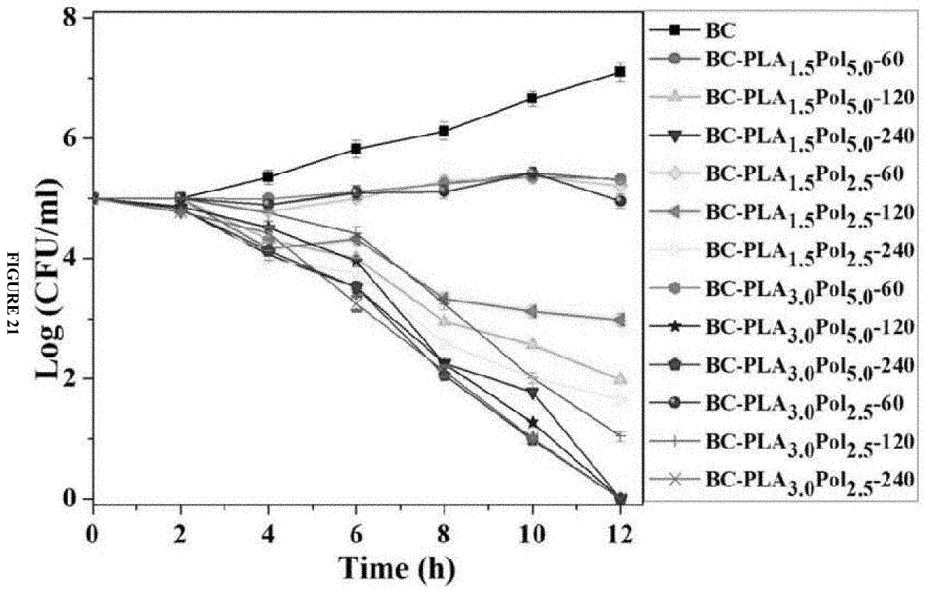

The anti-fungal activity of the microcapsules and cellulosic matrices were checked for inhibition of Candida albicans. The studies are depicted in FIG. 15, FIG. 16, FIG. 17 and FIG. 18.

TABLE 7
Antifungal activity of microcapsules and bacterial
cellulose loaded microcapsules

| | | Zone of inhibition | | |
|---|---|---|---|---|
| Sample | Code | 24 hrs | 48 hrs | 72 hrs |
| BC-PLA_{1.5}-Pol_{5.0} | a | 4.3 ± 0.2 | 3.6 ± 0.1 | 2.5 ± 0.2 |
| BC-PLA_{1.5}-Pol_{2.5} | b | 3.9 ± 0.1 | 3.1 ± 0.1 | 2.1 ± 0.2 |
| BC-PLA_{3.0}-Pol_{5.0} | c | 3.9 ± 0.1 | 3.7 ± 0.1 | 2.9 ± 0.2 |
| BC-PLA_{3.0}-Pol_{2.5} | d | 4.1 ± 0.2 | 3.4 ± 0.2 | 2.4 ± 0.3 |
| PLA_{1.5}-Pol_{5.0} | a′ | 4.6 ± 0.2 | 2.7 ± 0.2 | 1.9 ± 0.1 |
| PLA_{1.5}-Pol_{2.5} | b′ | 4.3 ± 0.1 | 1.6 ± 0.1 | 1.4 ± 0.1 |
| PLA_{3.0}-Pol_{5.0} | c′ | 4.2 ± 0.1 | 2.5 ± 0.2 | 1.8 ± 0.1 |
| PLA_{3.0}-Pol_{2.5} | d′ | 3.9 ± 0.1 | 1.9 ± 0.1 | 1.6 ± 0.1 |
| Positive control | e | 3.5 ± 0.1 | 1.8 ± 0.1 | 1.5 ± 0.1 |
| (Fluconazole) | | | | |
| Negative control | f | No zone | No zone | No zone |

FIG. 15 shows the antifungal activity of the microcapsule loaded cellulosic matrices and microcapsules after 24 hrs of incubation. FIG. 16 shows the antifungal activity of the microcapsule loaded cellulosic matrices and microcapsules after 48 hrs of incubation. FIG. 17 shows antifungal activity of the microcapsule loaded cellulosic matrices and microcapsules after 72 hrs of incubation. FIG. 18 shows antifungal activity of the positive and negative controls used in the studies.

It is clearly exhibited that both microcapsules, as well as loaded cellulosic matrices, show anti-fungal activity and the inhibition is higher than the positive control.

### Example 12: Time-Kill Curve Analysis of Oil Compositions, Microcapsules and Bacterial Cellulose Loaded Microcapsules

Time kill curve analysis was done to determine the efficacy of the oil compositions, microcapsules and bacterial cellulose loaded microcapsules.

In the first study, time-kill plot of mean values for log₁₀ of the numbers of CFU/milliliter versus time for C. albicans tested against Fluconazole (64, 128 and 256 μg/ml as positive control), Thymol (120 and 240 μg/ml), Eugenol (120 and 240 μg/ml) and Carvacrol (120 and 240 μg/ml) and combination of three oils 120 μg/ml (40+40+404 ml) and 240 μg/ml (80+80+80 μg/ml) and, Drug free tube (as negative control). The results are depicted in FIG. 19.

It is clearly exhibited that the combination of three oils shows more reduction in number of colony-forming units per mL than the individual oils, which confirms the synergistic activity exhibited by combination of oils. Further, the oil compositions show better activity than that of the fluconazole (standard control drug). The combination of oils shows 99.9% reduction in growth after 10 h and after 12 h for individual oils at 240 μg/ml (2 MIC), which again confirms the synergistic effect of the oil compositions.

In the second study, time-kill plot of mean values for log₁₀ of the numbers of CFU/milliliter versus time for C. albicans tested against PLA_{1.5}-Pol_{5.0}, PLA_{1.5}-Pol_{2.5}, PLA_{3.0}-Pol_{5.0}, PLA_{3.0}-Pol_{2.5} (60, 120 and 240 μg/ml which corresponds to 0.5, 1 and 2 MIC respectively) and pure bacterial cellulose (as negative control). The results are depicted in FIG. 20. Most of the microcapsules with exhibit fungicidal activity which exhibits the efficacious nature of the microcapsules.

In the third study, time-kill plot of mean values for log₁₀ of the numbers of CFU/milliliter versus time for C. albicans tested against BC-PLA_{1.5}-Pol_{5.0}, BC-PLA_{1.5}-Pol_{2.5}, BC-PLA_{3.0}-Pol_{5.0}, BC-PLA_{3.0}-Pol_{2.5} (60, 120 and 240 μg/ml which corresponds to 0.5, 1 and 2 MIC respectively) and pure bacterial cellulose (as negative control). The results are depicted in FIG. 21. Most of the loaded bacterial cellulosic matrices exhibit fungicidal activity which exhibits the efficacious nature of the matrices.

The studies exhibit that the oil compositions, microcapsules and bacterial cellulose loaded microcapsules are highly efficacious in inhibiting fungal growth.

### Advantages of the Present Invention

The present composite could work as an excellent panty liner for feminine hygiene purpose as the bacterial cellulose loaded microcapsules (MC) absorbs the excess exudates from the vaginal cavity, non-irritant to the vaginal mucosa. MC also assists in the sustained release of oil from the microcapsules. The composite works well as a mucoadhesive patch for the delivery of drugs in the buccal and oral cavity, as the MC gets rehydrated quickly and the chitosan of the optimum degree of deacetylation has mucoadhesive properties. The same composite works well as chewing gum as the MC itself has high resilience, water-absorbing capability and the nanofibrous hydrogel nature. MC if swallowed could be excreted in the feces and it is generally regarded as safe (GRAS).

The compositions and medicated cellulosic matrix, modified as chewing gum for oral candidiasis do not require water to swallow and hence, advantageous for patients having difficulty in swallowing. The cellulosic matrices are highly acceptable by children. The matrices are also helpful for prevention and treatment of gum inflammation and tooth decay. Further, the first-pass metabolism is avoided and increase in the bioavailability of drugs at the site of infection enhances the therapeutic effects.

Similarly, the medicated cellulosic matrices for prevention and treatment of vaginal candidiasis bypasses the first-pass metabolism which results in greater bioavailability. Further, the delivery systems result in reduction in the incidence and severity of gastrointestinal side effects. Finally, the medicated matrix of the invention fulfills several consumer preferences such as being odorless and colorless, causing no irritation, itching, burning or swelling, low dosage frequency, high retention time and convenience of self-application.

We claim:

1. A composition comprising at least two oils selected from a group comprising thymol oil, eugenol oil and carvacrol oil.

2. The composition as claimed in claim 1, wherein the oils are present at a volume/volume ratio in the range from 1:5 to 5:1.

3. The composition as claimed in claim 1, wherein the composition comprises thymol oil, eugenol oil and carvacrol oil.

4. The composition as claimed in claim 3, wherein each oil is present in the composition at a volume/volume ratio in the range from 1:10 to 10:1.

5. The composition as claimed in claim 1 comprising one or more pharmaceutically acceptable carrier, excipient or preservatives.

6. A microcapsule composition, comprising a shell material and a core material, wherein the core material of the microcapsule comprises a composition as claimed in claim 1.

7. The microcapsule composition as claimed in claim 6, wherein the shell material in the microcapsule is selected from a group comprising polylactide (PLA), chitosan, Polymethyl methacrylate (PMMA), poly(N-isopropylmethacrylamide) (PNIPAM) and aliginates.

8. The microcapsule composition as claimed in claim 6, prepared by a process comprising the steps of:
a. preparing an emulsion comprising oil composition as claimed in claim 1, a surfactant and water;
b. adding a polymer solution and a cross-linker to the emulsion prepared in step (a) to obtain the microcapsule composition.

9. The microcapsule composition as claimed in claim 8, wherein the surfactant is selected from a group comprising Poloxamer 188, Tween 20 and Tween 80.

10. The microcapsule composition as claimed in claim 8, wherein the cross-linker is selected from a group comprising Octamethyl cyclo tetra siloxane (OCMTS), glutaraldehyde, TPP (Sodium Tripolyphosphate), NaOH and genipin.

11. A microbial cellulosic matrix comprising microcapsule composition as claimed in claim 6.

12. The microbial cellulosic matrix as claimed in claim 11, wherein the cellulosic matrix is derived from a bacterial species selected from a group comprising Gluconacetobacter xylinus, Gluconacetobacter hansenii, Acetobacter xylinus, Acetobacter xylinum, or mutants or genetic variants thereof.

13. The microbial cellulosic matrix as claimed in claim 11, wherein the cellulosic matrix is prepared by incubating freeze dried cellulosic matrix with the microcapsule composition.

14. The composition as claimed in claim 1, microcapsule composition as claimed in claim 6 or microbial cellulosic matrix as claimed in claim 14 for use in the prevention or treatment of candidiasis.

15. The microcapsule composition as claimed in claim 6 or microbial cellulosic matrix as claimed in claim 14 for use in preparation of chewing gums, mucoadhesive patches, transdermal patches and liners.
